(19) United States
(12) Patent Application Publication
(10) Pub. No.: US 2017/0122949 A1
(43) Pub. Date: May 4, 2017

(54) DIAGNOSIS AND TREATMENT OF CILIATED HEPATIC FOREGUT CYSTS

(71) Applicant: Kiromic, LLC, Lubbock, TX (US)

(72) Inventors: Maurizio CHIRIVA-INTERNATI, Lubbock, TX (US); Scott DAHLBECK, Lubbock, TX (US)

(73) Assignee: Kiromic, LLC, Lubbock, TX (US)

(21) Appl. No.: 15/330,444

(22) PCT Filed: Mar. 23, 2015
(86) PCT No.: PCT/US15/22069

§ 371 Date: Sep. 21, 2016

Related U.S. Application Data
(60) Provisional application No. 61/968,445, filed on Mar. 21, 2014.

(51) Int. Cl. G01N 33/574; A61B 17/32; A61B 10/02; C12Q 1/68

(52) U.S. Cl. CPC G01N 33/57438; C12Q 1/6886; A61B 17/320016; A61B 10/0283; C12Q 2600/158

(57) ABSTRACT

This invention provides methods and compositions for diagnosis and treatment of ciliated hepatic foregut cysts (CHFC). In particular, the methods utilize one or more cancer-testis (CT) antigens as a marker for CHFC. The methods can involve the following steps: (a) obtaining a biological sample from a subject; (b) measuring the level of a CT antigen (or the level of the CT antigen nucleic acid such as mRNA or DNA) in the sample, and comparing the sample CT antigen level (or the level of the CT antigen nucleic acid such as mRNA or DNA) to the level in normal healthy subjects. A sample CT antigen (or its mRNA or DNA) level in excess of the level in normal healthy subjects indicates CHFC.

### CROSS REFERENCE TO RELATED APPLICATION

This application claims priority to U.S. Provisional Application No. 61/968,445 filed on Mar. 21, 2014, which is incorporated herein by reference in its entirety.

### FIELD OF THE INVENTION

The present invention relates to methods and compositions for the diagnosis and treatment of ciliated hepatic foregut cysts (CHFC) and other conditions. In particular, the present invention relates to detecting specific antigens to diagnose CHFC.

### BACKGROUND OF THE INVENTION

Ciliated hepatic foregut cysts (CHFCs) are rare, typically benign liver lesions of congenital origin. CHFCs are lined by a layer of ciliated columnar cells and containing mucoid material and debris [1, 2]. Originally described by Nikolaus Friedreich in 1857 [3], these lesions were termed CHFCs by Wheeler and Edmondson in 1984 [4] to describe solitary unilocular cysts of the liver with features distinct from biliary cystadenoma, and histologically similar to previously described ciliated bronchial and esophageal cysts. Histologically, CHFCs consist of four layers: ciliated pseudostratified columnar epithelium with interspersed goblet cells, loose sub-epithelial connective tissue, 1 to 3 smooth muscle layers, and an outer fibrous capsule [5].

CHFCs are generally found incidentally on radiological imaging [6, 7] or during surgical exploration [8]. They occur more frequently in men and are found most commonly in the medial segment of the left hepatic lobe, unlike most other solitary cysts that show a female predominance and greater occurrence in the right hepatic lobe. There has been an increase in the reported number of CHFC during the past 15 years [2]. This may reflect improved techniques, use and availability of even more sophisticated radiological imaging. Since 1988 there was an increase in the number of CHFCs recognized and described in the Japanese population, but the significance of this finding is still unclear [9, 10].

CHFCs are thought to develop either from a detached out-pouching of the hepatic foregut diverticulum or from abnormal bronchiolar budding [11]. Although CHFCs are retained benign lesions, a case of squamous cell metaplasia [12] and three cases of squamous cell carcinoma arising from a CHFC have been described [13-15].

The potential of malignant transformation, with its possibility of poor prognosis, makes necessary the identification of new biomarkers for more accurate diagnosis and treatment of CHFC.

### SUMMARY

The present invention provides for a method for diagnosing ciliated hepatic foregut cysts (CHFC) in a subject comprising the steps of: (a) obtaining a sample from the subject; (b) detecting in the sample the presence or absence of a cancer-testis (CT) antigen, its mRNA or its antibodies; and (d) diagnosing CHFC if the presence of a CT antigen or its mRNA (or antibodies) is detected.

The present invention also provides for a method for diagnosing ciliated hepatic foregut cysts (CHFC) in a subject comprising the steps of: (a) obtaining a sample from the subject; (b) measuring the level of a CT antigen, or the level of a CT antigen mRNA or its antibodies, in the sample; (c) comparing the CT antigen level in the sample, or the level of the CT antigen mRNA or its antibodies in the sample, to the level in a control sample; and (d) diagnosing CHFC if the CT antigen level in the sample, or the level of the CT antigen mRNA or its antibodies in the sample, is at least 120%, at least 150%, at least 200%, at least 250%, at least 300%, at least 350%, at least 400%, at least 450%, at least 500% of the level in the control sample. The control sample may contain one or more samples from normal healthy subjects.

Also encompassed by the present invention is a method for diagnosing and treating ciliated hepatic foregut cysts (CHFC) in a subject comprising the steps of: (a) obtaining a sample from the subject; (b) detecting in the sample the presence or absence of a CT antigen, its mRNA or its antibodies; and (c) treating CHFC in the subject if the CT antigen, or its mRNA or its antibodies, is detected.

Also provided is a method for treating ciliated hepatic foregut cysts (CHFC) in a subject comprising the steps of: (a) requesting a test providing the results of an analysis to determine whether the subject expresses a CT antigen in a sample, or the CT antigen level in the sample (or the level of the CT antigen mRNA or its antibodies in the sample), compared to the level in a control sample; and (b) treating CHFC in the subject if the CT antigen is expressed in the sample, or if the CT antigen level (or its mRNA or its antibodies level) is significantly higher than that in a control sample.

In step (b) of the methods described herein, an antibody or a fragment thereof specific to a CT antigen may be applied to the sample to detect the presence or absence of a CT antigen, or to measure the level of a CT antigen. The detection of the antibody-CT antigen complex, or the significantly higher level of the antibody-CT antigen complex, may indicate CHFC. The antibody or a fragment thereof may be used in an immunohistochemistry or immunofluorescence assay.

The present invention additionally provides for a method for diagnosing ciliated hepatic foregut cysts (CHFC) in a subject comprising the steps of: (a) obtaining a sample from the subject; (b) applying to the sample an antibody or a fragment thereof specific for a cancer-testis (CT) antigen, wherein presence of the CT antigen creates an antibody-CT antigen complex; (c) detecting the antibody-CT antigen complex; and (d) diagnosing CHFC where the antibody-CT antigen complex is detected.

In one embodiment, the CT antigen is Sperm protein 17 (Sp17), AKAP-associated sperm protein (ASP), or a combination thereof. In another embodiment, the CT antigen is SP17, ASP, NY-ESO-1, CABYR, TSP50, BORIS, RQCD1, BAGE, SSX, SCP-1, Piwi12, LAGE-1, SSX, AKAP, SCP-1, or combinations thereof.

The sample may be a tissue, cells, a body fluid such as blood, serum, plasma etc.

When CHFC is diagnosed in the subject, it may be treated. Treatment may involve removing the cyst(s) by surgery (e.g., laparoscopic excision) or administering to the subject an agent that attenuates the CT antigen's level or activity. In one embodiment, the agent is an antibody or a fragment thereof, an siRNA, an shRNA, an antisense molecule, or a ribozyme. The agent may be administered systemically, or injected into a cyst(s).

The subject may be additionally or preliminarily diagnosed by ultrasound, computed-tomography (CT), magnetic resonance imaging (MRI), fine needle aspiration cytology (FNAC), immunohistochemistry, surgery, or a combination thereof.

Also encompassed by the present invention is a method for diagnosing ciliated hepatic foregut cysts (CHFC) in a subject comprising the steps of: (a) obtaining a sample from the subject; (b) detecting in the sample the presence or absence of Sperm protein 17 (Sp17) and/or AKAP-associated sperm protein (ASP); and (c) diagnosing CHFC if SP17 and/or ASP are detected. In one embodiment, SP17 and ASP are detected by antibodies specific to SP17 and ASP, respectively.

### BRIEF DESCRIPTION OF THE FIGURES

FIG. 1. Western blots of spermatozoa obtained from healthy, fertile donors' lysates, which demonstrate the specificity of the antibodies against Sperm protein 17 (Sp17) and AKAP-associated sperm protein (ASP), respectively on the labeled histological sections. β-actin shows an immunoreactive band of 42 kDa. The reference molecular weights are indicated at left.

FIG. 2. Sp17 (A) and ASP (B) were localized within the cytoplasm of ciliated cells lining the cysts, and in their cilia. No immunoreactivity was recognized at the level of sub-epithelial connective tissue, smooth muscle layer, or of the outer fibrous capsule. Objective magnification 20×. Inset of FIG. 2B, objective magnification 40×.

FIG. 3. Confocal microscopy demonstrates that both Sp17 and ASP overlap in the same region of the cell. No immunoreactivity was recognized at the level of sub-epithelial connective tissue, smooth muscle layer, or of the outer fibrous capsule, confirming the immunohistochemistry analysis. Panel A, objective magnification, 20×; Panel B, objective magnification, 40×.

### DETAILED DESCRIPTION

This invention provides methods and compositions for diagnosis and treatment of CHFC. In particular, the methods utilize one or more cancer-testis (CT) antigens as a marker for CHFC. The methods may involve the following steps: obtaining a biological sample from a subject; measuring the level of a CT antigen (or the level of the CT antigen nucleic acid such as mRNA or DNA) in the sample, and comparing the sample CT antigen level (or the level of the CT antigen nucleic acid such as mRNA or DNA) to the level in a control (e.g., from one or more normal healthy subjects). A sample CT antigen (or its mRNA or DNA) level in excess of the level in the control indicates CHFC.

### Cancer-Testis (CT) Antigens

Cancer-testis (CT) antigens are proteins expressed in normal gametogenic tissues and in different types of tumors (Scanlan et al., 2002, Immunol Rev. 188:22-32. Scanlan et al., 2004, Cancer Immun. 4:1. Zendman et al., 2003, J Cell Physiol. 194(3):272-88. Simpson et al., 2005, Nat Rev Cancer. 5(8):615-25. Bodey, 2002, Expert Opin Biol Ther. 2(6):577-84). In testis, CT antigens are expressed exclusively in cells of the germ cell lineage, although there is a marked variation in the protein expression pattern during different stages of sperm development. Likewise, a heterogeneous expression is also observed in tumors. It has been proposed that the aberrant expression of CT antigens in tumors recapitulates portions of the germline gene expression program and is related to some characteristics of the neoplastic phenotype such as immortality, invasiveness, immune evasion and metastatic capacity. Chiriva-Internati et al., Cancer Testis Antigens: A Novel Target in Lung Cancer, International Reviews of Immunology, 31:321-343, 2012. Pandey et al., Cancer Testes Antigens in Breast Cancer: Biological Role, Regulation, and Therapeutic Applicability, International Reviews of Immunology, 31:302-320, 2012. Mirandola et al., Cancer Testis Antigens: Novel Biomarkers and Targetable Proteins for Ovarian Cancer, International Reviews of Immunology, 30:127-137, 2011. Simpson et al., 2005, Nat Rev Cancer. 5(8):615-25. Old, L J., 2001, Cancer Immun. 1:1. U.S. Pat. No. 8,207,300.

Non-limiting examples of CT antigens include SP17, ASP, NY-ESO (NY-ESO-1, etc.) CABYR, TSP50, BORIS, RQCD1, BAGE, SSX, SCP-1, Piwi12, OY-TES-1, LAGE-1, AKAP, SCP-1/HOM-TES-14, MAGE (MAGE-A, MAGE-B, MAGE-C, etc.), GAGE (GAGE-A, GAGE-B, etc.), PAGE, XAGE, CAGE, HOM-TES-85, SAGE, BAGE, CT9/3BRDT, HAGE, SPO11, and SPAG9.

SP17 has prominent expression in cancers of various histological-type, such as melanoma, myeloma, ovarian, esophageal and lung cancer. In 2011, Chiriva et al. discovered expression of SP17, along with other CT antigens such as AKAP-4 and PTTG in NSCLC. Chiriva et al. Identification of new cancer/testis antigens in non-small cell lung cancer. J Immunol. 2011; 165:16. The SP17 N-terminus shares 45% similar identity with the human type II regulatory subunit of protein kinase A (RII alpha), while SP17 C-terminus is 43% identical to the calmodulin-binding site of neuromodulin. Chiriva-Internati M. Sperm protein 17: clinical relevance of a cancer/testis antigen, from contraception to cancer immunotherapy, and beyond. Int. Rev. Immunol. 2011; 30(2-3):138-149. Kong et al., Sequence and localization of the mouse sperm auto antigenic protein, Sp17. Biol. Reprod. 1995; 53(3):579-590. SP17 is crucial for spermatogenesis and its C-terminus functions as calmodulin, once it is truncated by proteases during the acrosome reaction. In addition, the N-terminus of SP17 participates in cAMP-dependent protein kinases including protein kinase A. SP17 also serves as an interacting molecule for A-kinase anchoring proteins (AKAP), a scaffold protein that delegates and localizes the PKA in different compartments within the cell. Lea et al., Association of sperm protein 17 with A-kinase anchoring protein 3 in flagella. Reprod. Biol. Endocrinol. 2004; 2(1):57. Recent studies have demonstrated that SP17 not only increases resistance to chemotherapy as seen in ovarian cancer and esophageal cancer but it also increases metastasis and results in poor prognosis. Chiriva-Internati et al., Tumor vaccine for ovarian carcinoma targeting sperm protein 17. Cancer 2002; 94(9):2447-2453. Chiriva-Internati et al. A NOD/SCID tumor model for human ovarian cancer that allows tracking of tumor progression through the biomarker Sp17. J. Immunol. Methods. 2007; 321(1-2):86-93. Kausar T. Sperm protein 17 is a novel marker for predicting cisplatin response in esophageal squamous cancer cell lines. Int. J. Cancer. 2010; 126(6):1494.

New York esophageal squamous cell carcinoma-1 (NY-ESO-1) is also known as CTAG1B or LAGE2b. Lee et al. studied NY-ESO-1 in cultured lung cancers and demonstrated that HLA-restricted cytotoxic T lymphocyte recognized NY-ESO-1 expression. Lee et al. NY-ESO-1 may be a potential target for lung cancer immunotherapy. Cancer J. Sci. Am. 1999; 5(1):20-25. Moreover, NY-ESO-1 is expressed in various cancers including lung, melanoma, myeloma, sarcoma, bladder, breast, and ovarian cancer. Juretic et al. Expression and possible prognostic role of MAGE-A4, NYESO-1, and HER-2 antigens in women with relapsing invasive ductal breast cancer: retrospective immunohistochemical study. Croat Med J. 2006; 47(1):32-41. Cho et al. Physical interaction of two cancer-testis antigens, MAGE-C1 (CT7) and NY-ESO-1 (CT6). Cancer Immun. 2006; 6:12. Sugita et al. NY-ESO-1 expression and immunogenicity in malignant and benign breast tumors. Cancer Res. 2004; 64(6):2199-2204.Theurillat et al. NY-ESO-1 protein expression in primary breast carcinoma and metastases—correlation with CD8+ T-cell and CD79a+ plasmacytic/B-cell infiltration. Int. J. Cancer. 2007; 120(11):2411-2417.

CABYR was historically seen in testis-specific tissue, but recently CABYR was observed to be expressed in brain tumors. Moreover, Lou et al. discovered expression of CABYR in cultured lung cancer cells that consisted of adenocarcinomas and squamous cell carcinomas. Luo et al., CABYR is a novel cancer-testis antigen in lung cancer. Clin. Cancer Res. 2007; 13(4):1288-1297. The same group concluded from the same study that CABYR also stimulates immunogenic responses, and therefore would be an excellent target for immunotherapy. The role of CABYR is suggested to be in sperm capacitation where it has observable calcium-binding capacity and may participate in calcium-signaling pathways that regulate sperm motility. Naaby-Hansen et al. CABYR, a novel calcium-binding tyrosine phosphorylation-regulated fibrous sheath protein Involved in capacitation. Dev Biol. 2002; 242(2):236-254. There are different isoforms of CABYR including CR-A and CR-B of which the calcium binding occurs only with CR-A. Almeida et al. CT database: a knowledge-base of high-throughput and curated data on cancer-testis antigens. Nucleic Acids Res. 2009; 37(Suppl. 1):D816-D819. Similar to SP17, the N-terminus of CABYR contains an RII dimerization domain, which allows binding of CABYR to AKAP3 and ropporin. Li et al. CABYR binds to AKAP3 and ropporin in the human sperm fibrous sheath. Asian J Androl. 2011; 13(2):266-274. Fibrous sheath CABYR-binding protein (FSCB), a novel protein kinase A-phosphorylated calcium-binding protein, is a binding partner of CABYR. FSCB is also a substrate for PKA phosphorylation, serine/threonine phosphorylation, cAMP, and cGMP-dependent protein kinase phosphorylation. Li et al. FSCB, a novel protein kinase A-phosphorylated calcium-binding protein, is a CABYR-binding partner involved in late steps of fibrous sheath biogenesis. J. Biol. Chem. 2007; 282(47):34104-34119.

Testes-specific protease (TSP50) represents a type of threonine protease and may have a crucial role in mammalian spermatogenesis. In 2011, TSP50's function was discovered to be associated with the nuclear factor κB (NF-κB) signaling pathway. Song et al. Testes-specific protease 50 (TSP50) promotes cell proliferation through the activation of the nuclear factor κB (NF-κB) signaling pathway. Biochem. J. 2011; 436(2):457-467. TSP50 has potent influence of the NF-κB pathway because it inhibits the inhibitor of NF-κB, also known as the IκBa complex. With this control over the NF-κB pathway, TSP50 can regulate tumor necrosis factor α (TNFα), the PMA-induced NF-κB (nuclear factor κB)-responsive reporter activity, and the p65 nuclear translocation. TSP50 is highly implicated in breast cancers due to its presence at both the mRNA level (28%) and protein level (92%). Shan et al. TSP50, a possible protease in human testes, is activated in breast cancer of epithelial cells. Cancer Res. 2002; 62(1):290-294. Melanocytic lesions and testicular cancers also show an increased TSP50 mRNA expression.

The Brother of the Regulator of Imprinted Sites (BORIS) gene is located at 20q13.31 and it functions as a significant transcriptional regulator by directing epigenetic reprogramming at CTCF sites in normal cells and tumor development. D'Arcy et al. BORIS, a paralogue of the transcription factor, CTCF, is aberrantly expressed in breast tumours. Br J Cancer. 2008; 98:571-579. It regulates the expression of MAGE-Al, NY-ESO-1, TSP50, and SPANX. Hong et al. Reciprocal binding of CTCF and BORIS to the NY-ESO-1 promoter coincides with de-repression of this cancer-testis gene in lung cancer cells. Cancer Res. 2005; 65:7763-7774. Vatolin et al. Conditional expression of the CTCF-paralogous transcriptional factor BORIS in normal cells results in demethylation and de-repression of MAGE-A1 and reactivation of other cancer-testis genes. Cancer Res. 2005; 65:7751-7762. Dougherty et al. established the fundamental role that BORIS plays in longevity of cancer cells when they demonstrated increased apoptosis in MDA-MB-231 breast cancer cells upon silencing of the BORIS gene. Dougherty et al. Selective apoptosis of breast cancer cells by siRNA targeting of BORIS. Biochem Res Commun. 2008; 370(1):109-112. BORIS's expression has been reported at the mRNA level in breast, gynecological, and melanocytic cancers and at the protein level in breast cancer.

RQCD1, located on 2q35, has been suggested to be involved in breast cancer development. It has been identified in a CCR4-NOT transcription complex that participates in mRNA transcription and degradation. Chen et al. Purification and characterization of the 1.0 MDa CCR4-NOT complex identifies two novel components of the complex. J. Mol. Biol. 2001; 14(4):683-694. In 2009, it was discovered that RQCD1 also interacts with the Grb10 interacting proteins 1 (GIGYF1) and 2 that play instrumental roles in aberrantly activating the Akt pathway in breast cancer cells. Ajiro et al. Involvement of RQCD1 overexpression, a novel cancer-testis antigen, in the Akt pathway in breast cancer cells. Int. J. Cancer. 2009; 35(4):673-681. Therefore, RQCD1 may have oncogenic activity because it promotes tumor growth by uncontrolled activation of the Akt pathway in breast cancer cells.

The BAGE gene, found on 21p11.1, has been found to be present in many cancers at the mRNA level; however, there is no information regarding its function.

Synovial Sarcoma X (SSX) was discovered in synovial sarcoma tissues and cell lines, as the fusion gene product SS18-SSX from the chromosomal translocation of t(X;18(p11.2;q11.2). Scanlan et al., The cancer/testis genes: Review, standardization, and commentary. Cancer Immun. 2004 Jan. 23; 4:1. Clark et al. Identification of novel genes, SYT and SSX, involved in the t(X;18)(p11.2;q11.2) translocation found in human synovial sarcoma. Nat. Genet. 1994; 7:502-508. These fusion gene products also encode for nuclear proteins that differ in transcriptional activities. While SS18 promotes transcription, SSX represses transcription and together they down-regulate COM1, a regulator of gene cell proliferation. SSX has been found to have immunogenic properties in melanoma, breast, colon, and ovarian cancer. So far, nine SSX genes have been identified and all are on chromosome X. Gure et al. The SSX gene family: Characterization of 9 complete genes. Int. J. Cancer 2002 Oct. 10; 101:448-453. Of this family, SSX-1, SSX-2, SSX-4, and SSX-5 are expressed at varying frequencies, whereas they are all expressed in the testis. The PCR analysis on OC patients and cell lines showed no correlation between the expression of SSX genes and clinic pathologic characteristics such as histological type, grade, recurrence, and survival. Valmori et al. Expression of synovial sarcoma X (SSX) antigens in epithelial ovarian cancer and identification of SSX-4 epitopes recognized by CD4+ T cells. Clin. Cancer Res. 2006 Jan. 15; 12:398-404. Furthermore, there is evidence of a correlation between a positive prognosis and the presence of spontaneous antibodies against SSX-2 and SSX-4, especially CD4+ T cells against specific epitopes of SSX-4.

HOM-TES-14 (SCP-1) is a synaptonemal complex protein involved in chromosomal reduction during meiosis (Tammela et al. SCP-1 cancer/testis antigen is a prognostic indicator and a candidate target for immunotherapy in epithelial ovarian cancer. Cancer Immun. 2004 Oct. 18; 4:10), and its expression was confirmed in different types of tumors. Meuwissen et al. A coiled-coil related protein specific for synapsed regions of meiotic prophase chromosomes. EMBO J 1992; 11:5091-5100. The analysis of the expression rate of this antigen was performed on different ovarian cell samples, tumoral OC (ovarian cancer) cell lines, normal ovary cell lines, and a panel of normal tissues. None of the ovarian cell lines, tumoral or normal, expressed SCP-1, while 15% of the specimens expressed it. Cannistra S A. Cancer of the ovary. N. Engl. J. Med. 2004 Dec. 9; 351:2519-2529. No expression of the gene was detectable either by immunohistochemical analysis or by ELISA for immunogenicity. However, data collected during the analysis about this antigen suggest a relationship between SCP-1 expression and survival in EOC (epithelial ovarian carcinoma). The median survival time of women SCP-1+ was significantly shorter than SCP-1− patients (25 months versus 97 months, p=0.004), which showed evidence of the possible fundamental role of this antigen in disease progression. Cannistra SA. Cancer of the ovary. N Engl J Med 2004 Dec. 9; 351:2519-2529. Expression of SCP-1 is seen in numerous cell lines including malignant gliomas, breast, renal cell, and epithelial ovarian cancer. In epithelial ovarian cancers, SCP-1 has been correlated with poor prognosis and survival. Cheng et al., Cancer/testis (CT) antigens, carcinogenesis and spermatogenesis. Spermatogenesis. 2011; 1(3):209-220.

OY-TES-1 is the human homologue of proacrosin binding protein sp32 precursor, identified in other mammal species, such as the pig and mouse. Baba et al., Sperm from mice carrying a targeted mutation of the acrosin gene can penetrate the oocyte zona pellucida and effect fertilization. J. Biol. Chem. 1994; 269:31845-31849.The expression of this CTA was studied and determined by reverse-transcription polymerase chain reaction (RT-PCR) in 100 EOC (epithelial ovarian carcinoma) samples, 5 EOC cell lines, and a panel of normal tissues. The same EOC tissues were analyzed by immunohystochemistry (IHC), and a sub-group of patients' sera was tested for antibodies against OY-TES-1. Although antibodies against this CT target were identified by ELISA in only 10% of the sera samples, the results of the RT-PCR and IHC confirmed the over-expression of this antigen in 69% of the tumor samples. Tammela et al., OY-TES-1 expression and serum immunoreactivity in epithelial ovarian cancer. Int. J. Oncol. 2006; 29:903-910. However, recent evidences show that OY-TES-1 is correlated with OC cell proliferation and accounts for paclitaxel resistance. Whitehurst et al., Tumor antigen acrosin binding protein normalizes mitotic spindle function to promote cancer cell proliferation. Cancer Res. 2010; 70:7652-7661.

Piwi genes are known to be the first gene class required for stem cell self-renewal in a large number of organisms. Cox et al. A novel class of evolutionarily conserved genes defined by piwi are essential for stem cell self-renewal. Genes Dev. 1998 Dec. 1; 12:3715-3727. Because of their importance in the self-renewal pathway of stem cells, gametogenesis and RNA interference (RNAi), these genes are highly conserved during evolution, and in mammals, these genes play a key role in spermatogenesis. Deng et al., a murine homolog of piwi, encodes a cytoplasmic protein essential for spermatogenesis. Dev. Cell 2002; 2:819-830. Kuramochi-Miyagawa et al. Mili, a mammalian member of piwi family gene, is essential for spermatogenesis. Development 2004; 131:839-849. RT-PCR analysis demonstrated the expression of Piwi12 gene only in the testis and its total absence in other normal tissues, both in mouse and human samples. Analysis of the expression of this gene by immunostaining using anti-Piwi12-antibody confirmed the presence of the protein only in the spermatogonia and spermatocytes in mouse and human samples. Kuramochi-Miyagawa et al. Two mouse piwi-related genes: Miwi andmili. Mech. Dev. 2001; 108:121-133. Lee et al. Stem-cell protein Piwi12 is widely expressed in tumors and inhibits apoptosis through activation of Stat3/Bcl -XL pathway. Hum. Mol. Genet. 2006 Jan. 15; 15:201-211. Sasaki et al. Identification of eight members of the Argonaute family in the human genome small star, filled. Genomics 2003; 82:323-330. Piwi12 activates the expression of Bcl-XL, a member of bcl-2 gene family. The bcl-2 gene family is involved in the regulation of cell death. Ke et al. Putative tumor suppressor Lats2 induces apoptosis through downregulation of Bcl-2 and Bclx(L). Exp. Cell Res. 2004 Aug. 15; 298:329-338. Reed J C. Bcl-2 family proteins. Oncogene 1998 Dec. 24; 17:3225-3236. ZamzamiN et al. Subcellular and submitochondrialmode of action of Bcl-2-like oncoproteins. Oncogene 1998 Apr. 30; 16:2265-2282. This suggests that inhibiting this gene could decrease the uncontrolled expansion of malignant cells. Results have recently been obtained showing that Piwi12 modulates chromatin modifications and enhances cisplatin resistance. Accordingly, Piwi12 knockdown in OC cell lines restore drug sensitivity. Wang et al., Stem cell protein Piwi12 modulates chromatin modifications upon cisplatin treatment. Mutat. Res. 2011; 14:14.

LAGE-1 gene encodes for a protein of 180 amino acid residues. Lethe et al. LAGE-1, a new gene with tumor specificity. Int. J. Cancer 1998 Jun. 10; 76:903-908. OC specimens were investigated by RT-PCR in order to evaluate LAGE-1 expression. It was found that LAGE-1 expression was detectable in 21% of the tumor specimens. In particular, the expression of LAGE-1 was analyzed in relation with NY-ESO-1 because of their high grade of homology, and considering both of the genes' expressions, about 50% of the specimens are positive for at least one of them. Odunsi et al. NY-ESO-1 and LAGE-1 cancer-testis antigens are potential targets for immunotherapy in epithelial ovarian cancer. Cancer Res 2003 Sep. 15; 63:6076-6083. There is a correlation between the expression of LAGE-1 and the later stages of disease, but there were no significant differences in the distribution of LAGE-1 expression and histological grade, disease-free survival and overall survival [43]. Analysis of patients' sera was performed in order to evaluate the presence of LAGE-1 antibodies. Because of the high homology with NY-ESO-1, it was impossible to separate the two specific antibodies so the data were correlated with the RT-PCR in order to correlate gene expression, stage, and histological characteristics. All of the positive patients (25%) had tumors of serous histology (except one with transitional cell carcinoma) and advanced stage. Odunsi et al. NY-ESO-1 and LAGE-1 cancer-testis antigens are potential targets for immunotherapy in epithelial ovarian cancer. Cancer Res 2003 Sep. 15; 63:6076-6083.

A-kinase anchoring proteins (AKAP) are a group of heterogeneous proteins that bind the regulatory subunit of protein kinase A (PKA) and enable PKA to be exposed to cAMP efficiently. Michel et al. AKAP mediated signal transduction. Annu Rev. Pharmacol. Toxicol. 2002; 42:235-257. Chemotherapy based on paclitaxel, which shows damage to microtubules, activates PKA and induces hyper-phosphorylation of Bcl-2 causing growth arrest and apoptosis, is common in OC treatment after surgery. Srivastava et al. Involvement of microtubules in the regulation of Bcl2 phosphorylation and apoptosis through cyclic AMP-dependent protein kinase. Mol. Cell. Biol. 1998; 18:3509-3517. Tortora et al. Synergistic inhibition of growth and induction of apoptosis by 8-chloro-cAMP and paclitaxel or cisplatin in human cancer cells. Cancer Res 1997 Nov. 15; 57:5107-5111. Covens et al. Systematic review of first-line chemotherapy for newly diagnosed post-operative patients with stage II, III, or IV epithelial ovarian cancer. Gynecol. Oncol. 2002 April; 85:71-80. Young et al., Paclitaxel: A pharmacoeconomic review of its use in the treatment of ovarian cancer. Pharmacoeconomics 2001; 19:1227-1259. The gene product of AKAP-3 is also involved in the regulation of human spermatozoa motility (Luconi et al., Cell Mol. Biol. (Noisy-le-grand) 2003; 49:357-369) and has been demonstrated to belong to sperm protein. Its mRNA expression has been observed only in the testis and normal human tissues (Vijayaraghavan et al. Isolation and molecular characterization of AKAP 110, a novel, sperm-specific protein kinase A-anchoring protein. Mol. Endocrinol. 1999; 13:705-717). AKAP-3 expression in OC and its relation with disease stage was confirmed in different studies. Hasegawa et al. A-kinase anchoring protein 3 messenger RNA expression in ovarian cancer and its implication on prognosis. Int. J. Cancer 2004 Jan. 1; 108:86-90. Sharma et al. A-kinase anchoring protein 3 messenger RNA expression correlates with poor prognosis in epithelial ovarian cancer. Gynecol Oncol 2005 October; 99:183-188. The presence of this antigen seems to be strictly correlated with a poor prognosis and with a low survival rate for the OC patients expressing AKAP-3. Tureci et al. Identification of ameiosis-specific protein as a member of the class of cancer/testis antigens. Proc. Natl. Acad. Sci. USA 1998 Apr. 28; 95:5211-5216.

### Diagnosis

Diagnostic methods of the invention can involve determining the aberrant expression of one or more of the CT antigen described herein, or the aberrant level of the corresponding nucleic acid molecules (e.g., CT antigen's mRNA, DNA that encode the CT antigen, etc.). The diagnostic methods of the invention can be used to detect the presence of a disorder (e.g., CHFC) associated with aberrant expression of a CT antigen (e.g., onset of the disorder), as well as to assess the progression and/or regression of the disorder, such as in response to a treatment.

In one embodiment, the present invention provides a method of diagnosing CHFC in a subject. The method may have the following steps: (a) obtaining a biological sample from the subject; (b) measuring the level of a CT antigen (or the level of mRNA or DNA of a CT antigen) in the sample and comparing the level to the CT antigen level (or the level of mRNA or DNA of a CT antigen) in a control (e.g., from a normal healthy subject(s)), where a statistically significant difference in CT antigen levels (or the level of mRNA or DNA of a CT antigen) indicates the presence of CHFC. The control may also be the level of the CT antigen in a disease-free control group or background levels measured in the same subject.

The diagnosis can be confirmed by correlation of the assay results with other clinical signs of disease known to those skilled in the clinical arts, such as the diagnostic modalities including, but not limited to, ultrasound, computed-tomography (CT), magnetic resonance imaging (MRI), fine needle aspiration cytology (FNAC), immunohistochemistry and surgery. Alternatively, the subject may have at least a preliminary diagnosis of CHFC based on the diagnostic modalities described herein.

The diagnosis indicated by the CT antigen level (or the level of mRNA or DNA of a CT antigen) measurements made according to the methods of the present invention can be independently confirmed with reference to clinical manifestations of disease known to practitioners of ordinary skill in the clinical arts.

The method for diagnosing a disorder (e.g., CHFC) characterized by aberrant expression of a CT antigen may involve: detecting expression of a CT antigen (or its mRNA) in a biological sample obtained from a subject, wherein aberrant level of a CT antigen or its mRNA (e.g., differential level of the CT antigen or its mRNA compared to a control) indicates that the subject has a disorder characterized by aberrant level of a CT antigen or its mRNA, such as CHFC.

As used herein, “aberrant” level of a CT antigen or its mRNA refers to any level that is significantly different from the expected (e.g., normal or baseline) level. For example, the level of a CT antigen (i.e., CT antigen polypeptides or the corresponding nucleic acid molecules such as DNA that encodes the CT antigen, or its mRNA) in a tissue or body fluid that is not expected to express the CT antigen would be included in the definition of aberrant level. Likewise, level of the CT antigen (or its mRNA or DNA) that is at a significantly higher or lower level than expected (e.g., in a control) is also included. Therefore, a determination of the level of one or more of the CT antigen and/or the CT antigen nucleic acid (e.g., mRNA, DNA etc.) is diagnostic of CHFC if the level is above or below a baseline level in a control sample(s) determined for that tissue type. The baseline level can be determined using standard methods known to those of skill in the art.

As used herein, the term “control” means predetermined values, and also means samples of materials tested in parallel with the experimental materials. Control samples include samples from control populations, biopsy samples taken from tissue adjacent to a biopsy sample suspected of CHFC, or control samples generated through manufacture to be tested in parallel with the experimental samples. Control samples may also mean a number of histologically normal tissue samples from subjects that are clinically normal (e.g., do not have clinical signs of CHFC) and determining the mean level of expression for the samples. Control samples may include positive and negative controls which may be a predetermined value that can take a variety of forms. The control(s) can be a single cut-off value, such as a median, mean or average. In one embodiment, the control is based on apparently healthy individuals that may be in an age bracket.

The level or amount of a CT antigen or its corresponding nucleic acid (DNA or mRNA, etc.) in a patient sample can be compared to a reference level or amount in a control sample. The control sample may be from a patient or patients with CHFC or a healthy subject or subjects. In other embodiments, a control sample is taken from a patient prior to treatment with a therapeutic intervention or a sample taken from an untreated patient. Reference levels can be determined by determining the level of a CT antigen or its corresponding nucleic acid (DNA or mRNA, etc.) in a sufficiently large number of samples obtained from normal, healthy control subjects to obtain a pre-determined reference or threshold value. A reference level can also be determined by determining the level of a CT antigen or its corresponding nucleic acid (DNA or mRNA, etc.) in a sample from a patient prior to treatment with the therapeutic intervention.

The level of a CT antigen or the CT antigen nucleic acid (mRNA, DNA, etc.) in a sample can indicate CHFC when the level is significantly higher or lower in the sample than in a control. In certain embodiments, a level in the sample that is at least about 105%, at least about 110%, at least about 120%, at least about 130%, at least about 140%, at least about 150%, at least about 160%, at least about 170%, at least about 180%, at least about 190%, at least about 200%, at least about 250%, at least about 300%, at least about 350%, at least about 400%, at least about 450%, at least about 500%, about 105%, about 110%, about 120%, about 130%, about 140%, about 150%, about 160%, about 170%, about 180%, about 190%, about 200%, about 250%, about 300%, about 350%, about 400%, about 450%, or about 500%, of the level in the control indicates CHFC.

In some embodiments, the steps of obtaining a biological sample and determining the level of a CT antigen or its corresponding nucleic acids in the sample may be repeated once or more times.

The invention also involves diagnosing or monitoring CHFC in a subject by determining the presence of an immune response to one or more CT antigens. This determination may be performed by assaying a tissue, cells or a body fluid from the subject for the level of antibodies against one or more CT antigens described herein. The presence of the antibodies against at least one CT antigen is indicative of the subject having CHFC.

In some embodiments, the step of determining the presence of the antibody includes contacting the biological sample with one or more CT antigen, and determining the binding of the antibody to the CT antigen. In one embodiment, the polypeptide is produced recombinantly and/or is bound to a substrate.

### Monitoring Disease Progression or Evaluation of Treatment Efficacy

The CT antigens can also be used to evaluate treatment efficacy (e.g. amelioration of one or more symptoms of a pathology). Where the amelioration of a disease (such as CHFC) can be related to reduction in levels of a CT antigen, the CT antigen levels in a biological assay sample taken from the patient can be measured before (for background), during and/or after (e.g., at a designated time, periodically or randomly) the course of treatment. The assay may be performed at regular intervals before and after each treatment.

The level of the CT antigen or its corresponding nucleic acid molecules, or the immune response against one of the CT antigens may be measured over time by sequential determinations. This permits monitoring of the disease and/or the effects of a course of treatment. For example, a first sample may be obtained from a subject, tested for level of a CT antigen or its corresponding DNA or mRNA, or an immune response to a CT antigen. At a second, subsequent time, a second sample may be obtained from the subject and similarly tested. The results of the first and second (or subsequent) tests can be compared as a measure of the onset, regression or progression of CHFC. If CHFC treatment was undertaken during the interval between obtaining the samples, the effectiveness of the treatment may be evaluated by comparing the results of the two tests. In certain embodiments, an increase in expression in the second sample compared to the first sample is indicative of onset or progression of CHFC, and a decrease in the expression in the second sample compared to the first sample is indicative of regression of CHFC.

In another embodiment, a method of monitoring effectiveness of CHFC treatment in a subject may have the following steps: (a) obtaining a first biological sample from a CHFC patient; (b) measuring the level of a CT antigen or its corresponding DNA or mRNA, or an immune response to a CT antigen, (c) providing one or more treatments of the CHFC; (d) obtaining a second biological sample from the CHFC patient during or after the one or more treatments; and (e) measuring a level of the CT antigen in the second biological sample and comparing the level of the CT antigen in the second sample to the level of the CT antigen in the first sample, wherein a lower level of the CT antigen in the second sample as compared to the CT antigen level in the first sample indicates efficacy of the treatment(s).

### Assays

In certain embodiments, the step of determining the level of the CT antigen or its nucleic acid molecules (mRNA, DNA, etc.) includes contacting the biological sample with an agent that selectively binds to the CT antigen or the nucleic acid. U.S. Pat. No. 7,670,599.

### Protein-Based Assays

The level of a CT antigen protein can be detected and/or quantified by any of a number of methods well known to those of skill in the art. The methods may include various immunoassays such as immunohistochemistry, enzyme-linked immunosorbent assay (ELISA), antibody sandwich capture assay, immunofluorescent assay, Western blot, enzyme-linked immunospot assay (EliSpot assay), precipitation reactions (in a fluid or gel), immunodiffusion, immunoelectrophoresis, radioimmunoassay (RIA), and the like. Also included are analytic biochemical methods such as electrophoresis, capillary electrophoresis, high performance liquid chromatography (HPLC), thin layer chromatography (TLC), hyperdiffusion chromatography, and the like. U.S. Pat. Nos. 4,366,241; 4,376,110; 4,517,288; and 4,837,168. Methods in Cell Biology Volume 37: Antibodies in Cell Biology, Asai, ed. Academic Press, Inc. New York (1993); Basic and Clinical Immunology 7th Edition, Stites & Terr, eds. (1991).

The level of a CT antigen may be detected by using molecules (e.g., polypeptides, etc.) that bind to the CT antigen. For example, the binding polypeptide may be an antibody or antibody fragment, such as an Fab, F(ab)₂, F(ab')₂, Fd, or Fv fragment of an antibody. Any of the various types of antibodies can be used for this purpose, including, but not limited to, polyclonal antibodies, monoclonal antibodies, humanized antibodies, human antibodies (e.g., generated using transgenic mice, etc.), single chain antibodies (e.g., single chain Fv (scFv) antibodies), heavy chain antibodies and chimeric antibodies. The antibodies can be from various species, such as rabbits, mice, rats, goats, chickens, guinea pigs, hamsters, horses, sheep, llamas etc.

The antibodies can be prepared by any suitable methods, including administering a protein, fragments of a protein, cells expressing the protein or fragments thereof and the like to an animal to induce polyclonal antibodies. The present invention also provides methods of producing monoclonal antibodies to the CT antigens described herein. The production of monoclonal antibodies is performed according to techniques known in the art.

In one embodiment, immunohistochemical methods are used to assay the CT antigen level. In one embodiment, antibodies that specifically bind to a CT antigen are contacted with a tissue sample (e.g., a histological sample). Those antibodies that specifically bind to the sample are visualized, or otherwise detected, and provide an indication of the location, presence, absence or quantity of the CT antigen in the sample. The antibodies are typically detected by detection of a label either affixed to the antibody or subsequently added after the tissue contacting step.

In another embodiment, Western blot (immunoblot) is used to detect and quantify a CT antigen in a sample. The technique may comprise separating sample proteins by gel electrophoresis, transferring the separated proteins to a suitable solid support, and incubating the sample with the antibodies that specifically bind the CT antigen.

The invention further includes protein microarrays (including antibody arrays) for the analysis of expression of CT antigens. Protein microarray technology, which is also known as protein chip technology and solid-phase protein array technology, is well known to those of ordinary skill in the art. Protein microarray may be based on, but not limited to, obtaining an array of identified peptides or proteins on a fixed substrate, binding target molecules or biological constituents to the peptides, and evaluating such binding. See, e.g., MacBeath et al., Printing Proteins as Microarrays for High-Throughput Function Determination, Science 289(5485):1760-1763, 2000. The tissue may be obtained from a subject or may be grown in culture (e.g., from a cell line). In some embodiments of the invention, one or more control peptide or protein molecules are attached to the substrate.

The polypeptides that may be used to assay the level of a CT antigen may be derived also from sources other than antibody technology. For example, such binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties. The CT antigens can be used to screen peptide libraries, including phage display libraries, to identify and select peptide binding partners of the CT antigens. Yeast two-hybrid screening methods also may be used to identify polypeptides that bind to the CT antigens.

### Nucleic Acid-Based Assays

The present methods may also assay the presence of or quantity the CT antigen gene or gene product. Gene products include nucleic acids (e.g. mRNAs) derived from the gene.

The level of the DNA or RNA (e.g., mRNA) molecules may be determined using routine methods known to those of ordinary skill in the art. The measurement result may be an absolute value or may be relative (e.g., relative to a reference oligonucleotide, relative to a reference mRNA, etc.). The level of the nucleic acid molecule may be determined by nucleic acid hybridization using a nucleic acid probe, or by nucleic acid amplification using one or more nucleic acid primers.

Nucleic acid hybridization can be performed using Southern blots, Northern blots, nucleic acid microarrays, etc.

For example, the DNA encoding a CT antigen in a sample may be evaluated by a Southern blot. Similarly, a Northern blot may be used to detect a CT antigen mRNA. In one embodiment, mRNA is isolated from a given cell sample, and then electrophoresed to separate the mRNA species. The mRNA is transferred from the gel to a solid support. Labeled probes are used to identify or quantity CT antigen nucleic acids.

In certain embodiments, labeled nucleic acids are used to detect hybridization. Complementary nucleic acids may be labeled by any one of several methods typically used to detect the presence of hybridized polynucleotides. One method of detection is the use of autoradiography. Other labels include ligands that bind to labeled antibodies, fluorophores, chemiluminescent agents, enzymes, and antibodies which can serve as specific binding pair members for a labeled ligand.

Nucleic acid microarray technology, which is also known as DNA chip technology, gene chip technology, and solid-phase nucleic acid array technology, may be based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP, etc.), hybridizing target nucleic acids to the probes, and evaluating target-probe hybridization. Jackson et al. (1996) Nature Biotechnology, 14: 1685-1691. Chee et al. (1995) Science, 274: 610-613.

The level of a CT antigen nucleic acid (e.g., RNA, DNA) may be assayed by in situ hybridization. Angerer et al. (1987) Methods Enzymol., 152: 649-660. In one embodiment, tissues or cells are denatured. The tissues or cells are then contacted with a hybridization solution at a moderate temperature to permit annealing of labeled probes specific to the CT antigen nucleic acids. The probes may be labeled with molecules as discussed herein.

The sensitivity of the hybridization assays may be enhanced through use of a nucleic acid amplification system that multiplies the target nucleic acid being detected.

Nucleic acid amplification assays include, but are not limited to, the polymerase chain reaction (PCR), reverse transcription polymerase chain reaction (RT-PCR), real-time RT-PCR, quantitative RT-PCR, etc.

Measuring or detecting the amount or level of mRNA in a sample can be performed in any manner known to one skilled in the art and such techniques for measuring or detecting the level of an mRNA are well known and can be readily employed. A variety of methods for detecting mRNAs have been described and may include, Northern blotting, microarrays, real-time PCR, RT-PCR, targeted RT-PCR, in situ hybridization, deep-sequencing, single-molecule direct RNA sequencing (RNAseq), bioluminescent methods, bioluminescent protein reassembly, BRET (bioluminescence resonance energy transfer)-based methods, fluorescence correlation spectroscopy and surface-enhanced Raman spectroscopy (Cissell, K. A. and Deo, S. K. (2009) Anal. Bioanal. Chem., 394:1109-1116).

The methods of the present invention may include the step of reverse transcribing RNA when assaying the level or amount of an mRNA.

These assays of determining the presence and/or level of the molecules of the invention in cells and tissues may include use of labels to monitor the presence of the molecules of the invention. The labels can be any material having a detectable physical or chemical property. Thus, a label is any composition detectable by spectroscopic, photochemical, biochemical, immunochemical, electrical, optical or chemical means. Such labels may include, but are not limited to, a fluorescent label, a radiolabel, a chemiluminescent label, an enzyme, a metallic label, a bioluminescent label, a chromophore, biotin etc. For example, a fluorescently labeled or radiolabeled antibody that selectively binds to a polypeptide of the invention may be contacted with a tissue or cell to visualize the polypeptide. In some aspects of the invention, a label may be a combination of the foregoing molecule types.

### Samples

As used herein, a sample includes, but is not limited to: tissue, cells, and/or body fluid (e.g., blood, plasma, serum, lymph node fluid, synovial fluid, cerebrospinal fluid, bronchial lavage, ascites fluid, bone marrow aspirate, pleural effusion, urine, etc.). The fluid sample may include cells and/or fluid. The tissue and cells may be obtained from a subject or may be grown in culture (e.g., from a cell line). Typically, a biological sample may be obtained by collecting a biopsy sample or a blood sample from a subject. In certain embodiments, a sample is taken from the tissue of interest.

### Kits

Also encompassed by the present invention are kits for use in the diagnostic applications described herein.

One example of the kits can be used for assaying the level of one or more CT antigens. The kits may include one or more of the following: assay reagents, buffers, CT antigen binding molecules (e.g. antibodies or fragments thereof, or other CT antigen binding molecules), and/or a suitable assay device.

The invention also includes kits for detection of a CT antigen nucleic acid (DNA or mRNA), including one or more of the following: assay reagents, buffers, probes and/or primers that can hybridize to the CT antigen nucleic acid (DNA or mRNA), a reverse transcriptase, and/or a suitable assay device.

Additional materials may be included in any or all kits of the invention, and such materials may include, but are not limited to buffers, water, enzymes, tubes, control molecules, etc. In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media may include addresses to internet sites that provide such instructional materials.

### Therapeutic Application

The present invention provides for methods of treating CHFC in a patient. These methods may involve the following steps: (a) obtaining a biological sample from a subject (the subject may or may not have a preliminary diagnosis of CHFC); (b) measuring a level of a CT antigen or CT antigen nucleic acid (DNA or mRNA) in the sample, and comparing the CT antigen level (or CT antigen nucleic acid level) in the sample to the CT antigen level (or CT antigen nucleic acid level) in a control (e.g., a sample(s) from normal healthy subjects), where a sample CT antigen level (or CT antigen nucleic acid level) in excess of CT antigen level (or CT antigen nucleic acid level) in a control indicates CHFC; and (c) treating the subject with CHFC.

Treatments for CHFC may include, but are not limited to: surgery, systemic therapies etc. The surgery may be performed to remove a cyst(s), through, e.g., laparoscopic excision. In one embodiment, treatment may include administering binding polypeptides such as antibodies or fragments thereof that specifically bind to one or more CT antigen. In another embodiment, the subject is treated with a molecule which attenuates the activity of at least one CT antigen. In a third embodiment, the treatment includes down-regulating CT antigen expression by RNA interference or RNAi (such as siRNAs, shRNAs, miRNAs, etc.), antisense molecules or ribozymes targeting CT antigen DNA or mRNA.

Administration may be by any enteral or parenteral route in dosages that will be varied by the skilled clinician depending on the patient's presenting condition. The administration may, for example, be oral, intravenous, intratumoral, intraperitoneal, intramuscular, intracavity, subcutaneous, or transdermal.

As will be clear to those skilled in the art, in some cases, the prognostic methods provided in the invention may be used in combination with other biomarkers of the disease. Accordingly, the methods described herein may be used, for example, in conjunction with other CHFC markers, diagnostic modalities including, but not limited to, ultrasound, computed-tomography (CT), MRI, fine needle aspiration cytology (FNAC), immunohistochemistry and surgery.

According to a further aspect of the invention, compositions containing the nucleic acid molecules, proteins, and/or binding polypeptides (e.g., antibodies or fragments thereof) of the invention are provided. The composition may also contain a pharmaceutically acceptable carrier. Pharmaceutically acceptable carriers include diluents, fillers, salts, buffers, stabilizers, solubilizers, and other materials which are well known in the art.

### Subjects

As used herein, a subject is preferably a human, non-human primate, cow, horse, pig, sheep, goat, dog, cat or rodent. In all embodiments, human subjects are preferred. In some embodiments, the subject is suspected of having CHFC or has been preliminarily diagnosed with CHFC.

The subjects may include humans or non-human mammals and therefore the methods encompass veterinary and/or livestock applications.

The following examples of specific aspects for carrying out the present invention are offered for illustrative purposes only, and are not intended to limit the scope of the present invention in any way.

### EXAMPLES

### Example 1

### Sperm Protein 17 (SP17) and AKAP-Associated Sperm Protein (ASP) are Expressed in Ciliated Hepatic Foregut Cysts

CHFCs have been described as benign and solitary cysts consisting of ciliated columnar epithelium [1], and containing mucoid material and debris. These cysts are usually unilocular and located on the anterior surface of the liver near the falciform ligament.

To date, only over 100 cases have been reported in the English and non-English literature [1]. CHFCs are typically found incidentally on radiological imaging or during surgical exploration. Despite its low incidence, an increase in the number of reports of CHFCs during the past 15 years has been noted, and is mainly associated to the availability of more accurate and sensitive radiological imaging modalities, including ultrasound and computed-tomography (CT) [1, 6, 7].

It has been shown that hepatic simple cysts are delimited by either columnar epithelium or mesothelial lining. Echinoccocal cysts lack lining epithelial cells and contain calicific debris, portions of hydatid cysts, or scolices. Epidermoid and endometrial cysts would contain squamous and endometrial epithelia, respectively [18]. The above features suggest that the lined ciliated epithelium is taken as the discriminating histological structure which distinguishes CHFCs from other hepatic cysts.

Additionally, the employment of different histochemical and immunohistochemical stains have shown that CHFCs produce mucin, stain positively for various cytokeratins and various other epithelial markers. Presence of smooth muscle has been identified in histology sections through pentachrome and smooth muscle actin stains [8,19].

CHFCs have generally been considered benign non-neoplastic lesions of developmental origin. However, Vick et al. in 1999 reported a squamous-cell carcinoma arising in a CHFC [15]. Since this first finding, other case reports have appeared with squamous-cell carcinomas arising in CHFCs, communicating with the gallbladder, involving the hepatic vein, causing portal hypertension in an adolescent, or presenting as solid masses on imaging studies. [10, 13-15, 20-22].

We investigated the immunohistochemical expression of Sperm protein 17 (Sp17) in four surgically resected CHFCs. Sp17, has been labeled as a cancer-testis (CT) antigen in multiple myeloma (mRNA encoding Sp17 has been found in 17% of patients) and in primary ovarian carcinoma (mRNA encoding Sp17 has been found in primary tumor cells from 70% of patients with primary ovarian carcinoma) [23-25]. It has also been found, at the protein level, in human germinal cells of the testis (except in the case of spermatogonia) [17], and in the ciliated epithelia of the respiratory airways and in both the male and female reproductive systems [26]. Sp17 has also been recognized in the synoviocytes of females affected by rheumatoid arthritis [27] and the melanophages of cutaneous melanocytic lesions [28], as well as in a proportion of primary nervous system tumors [29] and as a subset of esthesioneuroblastomas [30].

We also investigated the immunohistochemical expression of AKAP-associated sperm protein (ASP) which has a shared N-terminal sequence with Sp17 [34].

### MATERIALS AND METHODS

### Tissue Specimens

CHFC specimens were taken from four patients: two patients who went to the Medical College of Wisconsin, Milwaukee, USA and two patients who went to the Fundación Jiménez Díaz, Madrid, Spain. The two U.S. patients include a male, 63 years-old, who was admitted to the hospital because of a mass in his left kidney, and a liver mass suspicious for metastatic disease. This liver mass was cystic and upon further analysis showed ciliated epithelial lining The second patient was a 45-year-old male, who was found to have a suspicious liver mass on an outside CT scan. At that time the presenting symptoms were right upper quadrant and flank pain. He was taken to the operating room for excision of this cystic liver mass. Intra-operatively, an ultrasound was made, and enucleation of a segment 4A 2 cm cystic mass was performed. Additionally, he was found to have an intra-hepatic gallbladder. Upon further analysis this liver mass showed ciliated epithelial lining.

### Histochemistry

Two-micrometer thick sections were stained with a freshly made hematoxylin & eosin solution (Diapath, Bergamo, Italy), and analyzed under a light microscope (Leica DM6000B, Milan, Italy) by the same hepatopathologist.

### Western Blotting

In order to investigate the immune-specificity of the antibodies employed in the present study, spermatozoa taken from fertile human donors were collected [17], washed with PBS and subsequently incubated in a freshly made lyses buffer (Tris-HCl 500 mM pH7.4, NaCl 150 mM, Triton 100× 0.1%) at 4° C. for 15 minutes. The protein concentration was estimated with DC Protein Assay kit (Bio-Rad, Milan, Italy). Cell lysates (40 μg) were electrophoresed on a 12% polyacrylamide gel (Bio-Rad) and transferred onto nitrocellulose membranes (Bio-Rad). After being blocked with PBS containing 5% skim milk and 0.1% Tween 20 for 30 min, membranes were incubated with the primary mouse anti-human Sp17 monoclonal antibody (BD Biosciences, NJ, USA), or rabbit anti-human ASP polyclonal antibody (Imgenex, San Diego, USA), and secondary antibodies (anti-mouse IgG or anti-rabbit IgG, Sigma, Milan, Italy) at room temperature for 2 hours and 1 hour, respectively. The bands were visualized with Opti-4CN Substrate kit (Bio-Rad). β-actin was used as the control protein.

### Immunohistochemistry

Two-micrometer thick sections were processed for immunohistochemistry. After deparaffination and rehydration, the sections were placed in a bath for antigen retrieval for 30 minutes at 98° C. in a EDTA 1 mM solution, incubated with 3% H₂O₂ for 15 min to quench endogenous peroxidase activity, and then either treated for 2 hours at room temperature with primary antibodies raised against Sp17 (mouse anti-human Sp17 monoclonal antibody, BD Biosciences) or for 1 hour with primary antibodies against ASP (rabbit anti-human ASP polyclonal antibody, Imgenex). Mouse IgG1 or rabbit Ig fraction (Dako, Milan, Italy) were used as negative controls. This was followed by 30 min incubation with the DAKO Envision system (Dako). 3,3′-diaminobenzidine tetrahydrochloride (Dako) was used as a chromogen to yield brown reaction products. The nuclei were lightly counterstained with hematoxylin solution (Diapath). Slides were analyzed under a light microscope (Leica DM6000B, Milan, Italy).

### Immunofluorescence

Two-micrometer thick sections were processed for dual immunofluorescence. After deparaffination and rehydration, the sections were placed in a bath for antigen retrieval for 30 min at 98° C. in a 1 mM EDTA solution, followed by cooling for 30 min at room temperature. Then slides were treated with primary antibodies raised against ASP (rabbit anti-human ASP polyclonal antibody, Imgenex) at room temperature for 1 hour. After washing, slides were incubated with FITC goat-anti rabbit IgG Conjugate (Sigma) for 30 min. This was followed by 1 hour incubation with the antibodies raised against Sp17 (mouse anti-human Sp17 monoclonal antibody, BD Biosciences) washing and then the slides were incubated for 30 min with Cy-5 rabbit-anti mouse IgG Conjugate (Zymed Laboratories). Slides were subsequently counterstained for 5 minutes with DAPI (Invitrogen, Eugene, USA), washed and cover slipped with Gel Mount Aqueous (Sigma). All antibodies were diluted with PBS lx supplemented with 0.1% BSA. Washings were performed with PBS1×.

Slides were observed with a Leica TCS SP5 confocal microscope and processed by Leica software (Leica, Milan, Italy). To avoid overlapping between fluorochrome spectra, images were acquired by sequential scanning

### RESULTS

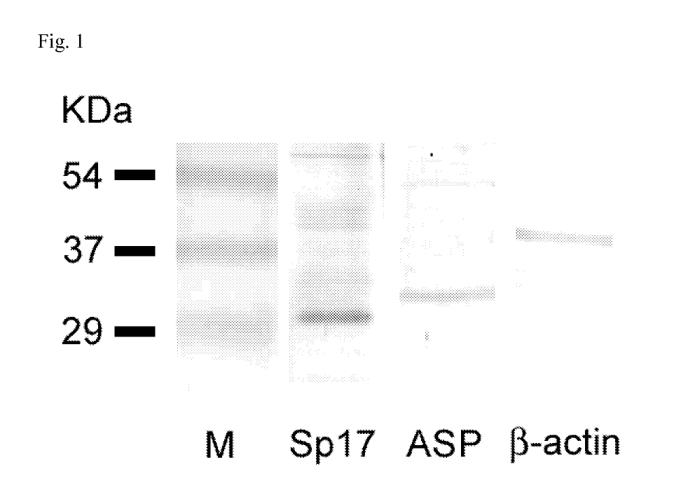

FIG. 1 shows Western blots of spermatozoa obtained from healthy, fertile donors' lysates, which demonstrate the specificity of the antibodies Sp17 and ASP on the labeled histological sections. β-actin shows an immunoreactive band of 42 kDa.

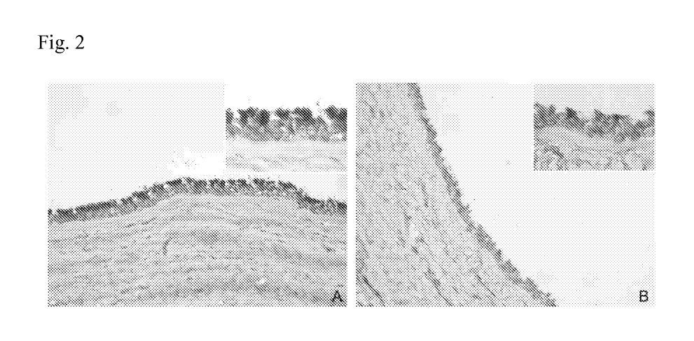

The immunohistochemistry experiments found that CHFCs immunopositive for Sp17 and ASP (FIG. 2). Both proteins were localized to the cytoplasm of ciliated cells lining the cysts, and in their cilia. No immunoreactivity was recognized at the level of subepithelial connective tissue, smooth muscle layer, or of the outer fibrous capsule.

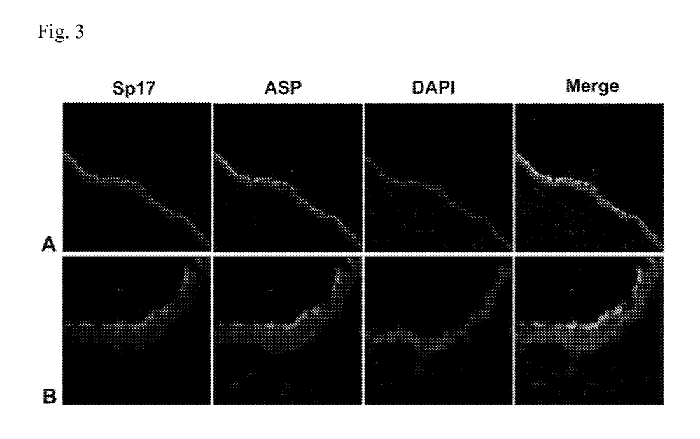

To verify whether Sp17 and ASP were co-expressed and co-localized within ciliated cells a dual immunofluoscence analysis was performed. Confocal microscopy demonstrates that both Sp17 and ASP overlap in the same region of the cell (FIG. 3). No immunoreactivity was recognized at the level of sub-epithelial connective tissue, smooth muscle layer, or of the outer fibrous capsule, confirming the immunohistochemistry.

The results obtained from all four CHFC specimens were identical. They were found immunopositive for Sp17 and ASP. Both proteins were localized to the cytoplasm of ciliated cells lining the cysts, and in their cilia. Confocal microscopy demonstrates that both Sp17 and ASP overlap in the same region of the cell.

### DISCUSSION

Sp17 and ASP cancer-testis antigens were found in ciliated cells of four CHFCs. Further characterization of Sp17 and ASP in CHFCs may provide significant clues for understanding the molecular mechanisms underlying their predisposition to develop squamous cell carcinomas. Grizzi et al., Sperm protein 17 and AKAP-associated sperm protein cancer-testis antigens are expressed in ciliated hepatic foregut cysts, Histopathology. 2015 Jan. 19. doi: 10.1111/his.12654.

The present study demonstrates the expression of Sp17 and ASP in ciliate cells lining the CHFCs, and their localization at the level of the cytoplasm and cilia (FIG. 2). Additionally, confocal microscopy has revealed the co-expression and co-localization of Sp17 and ASP in CHFCs ciliated epithelium (FIG. 3).

The expression of Sp17 and ASP in CHFCs suggest an important role of these two proteins in the development of these cysts.

Although CHFCs are retained benign lesions, one case of squamous cell metaplasia [12] and three cases of squamous cell carcinoma arising from CHFCs have been reported [13-15]. Interestingly, Sp17 has been detected in oral [43] and esophageal squamous cell carcinomas [44], and as a potential factor in the malignant transformation and metastatic tumor progression of murine squamous cell carcinoma [45]. It has been claimed that the identification of novel differentially expressed genes in these tumors adds to the repertoire of genes associated with murine and human squamous cell carcinogenesis and provides candidate potential biological targets for diagnosis and/or therapy [43, 44].

One of the most used tumor markers in biological fluids associated with cancer is the carbohydrate antigen 19-9 (CA 19-9). Several Authors have proposed CA 19-9 for detecting and discriminating benign from malignant CHFCs [1, 15, 43, 44]. However, all these studies concluded that although a raised level of this biomarker was detected, it was not useful because high levels have been also observed in benign CHFCs. As a consequence, aspiration cytology is today considered a primary technique in making a preliminary diagnosis of CHFCs, although it has been reported that this technique may miss malignant transformation through sampling error [1, 15, 46]. Sp17 and ASP expression in biological fluids may be tested in patients with a suspicion of CHFCs. Serum anti-Sp17 antibodies have been demonstrated at high levels in cancer patients. Gupta et al. have shown that circulating levels of anti-Sp17 antibodies, determined by ELISA, were significantly elevated in esophageal squamous cell carcinomas patients when compared with normal subjects [45]. Additionally, the development of a specific radioimmunoassay for Sp17 may be a valid tool for assessing its native distribution and aberrant expression.

Although the function of Sp17 is still unknown, the high degree of sequence conservation throughout its N-terminal half, and the presence of an A-kinase anchoring protein (AKAP)-binding motif within this region, suggests that it may play a regulatory role in a protein kinase A (PKA)-independent AKAP complex in both germinal and somatic cells [34-36].

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

### REFERENCES

1. Kaplan K J, Escobar M, Alonzo M, et al. Ciliated hepatic foregut cyst: report of a case on fine-needle aspiration. Diagn Cytopathol 2007; 35: 245-9.
2. Sharma S, Corn A, Kohli V, et al. Ciliated Hepatic Foregut Cyst: An Increasingly Diagnosed Condition. Dig Dis Sci 2008 Feb. 29. [Epub ahead of print]
3. Friedreich N. Cyst mit Flimmerepithel in der Leber. Arch Pathol Anat 1857; 11: 466-69.
4. Wheeler D A, Edmondson H A. Ciliated hepatic foregut cyst. Am J Surg Pathol 1984; 8: 467-70.
5. Bogner B, Hegedûs G. Ciliated hepatic foregut cyst. Pathol Oncol Res 2002; 8: 278-9.
6. Kadoya M, Matsui O, Nakanuma Y, et al. Ciliated hepatic foregut cyst: radiologic features. Radiology 1990; 175: 475-7.
7. Fang S H, Dong D J, Zhang S Z. Imaging features of ciliated hepatic foregut cyst. World J Gastroenterol 2005; 11: 4287-9.
8. Vick D J, Goodman Z D, Deavers M T, et al. Ciliated hepatic foregut cyst: a study of six cases and review of the literature. Am J Surg Pathol 1999; 23: 671-7.
9. Mannami T, Ohfuji Y, Fujiwara K, et al. Ciliated hepatic foregut cyst: report of a case and a summary of the 24 cases reported in Japan. Nippon Shokakibyo Gakkai Zasshi 2008; 105: 235-43.
10. Jakowski J D, Lucas J G, Seth S, et al. Ciliated hepatic foregut cyst: a rare but increasingly reported liver cyst. Ann Diagn Pathol 2004; 8: 342-6.
11. Stringer M D, Jones M O, Woodley H, et al. Ciliated hepatic foregut cyst. J Pediatr Surg 2006; 41: 1180-3.
12. Ben Mena N, Zalinski S, Svrcek M, et al. Ciliated hepatic foregut cyst with extensive squamous metaplasia: report of a case. Virchows Arch 2006; 449: 730-3.
13. de Lajarte-Thirouard A S, Rioux-Leclercq N, Boudjema K, et al. Squamous cell carcinoma arising in a hepatic forgut cyst. Pathol Res Pract 2002; 198: 697-700.
14. Furlanetto A, Dei Tos AP. Squamous cell carcinoma arising in a ciliated hepatic foregut cyst. Virchows Arch 2002; 441: 296-8.
15. Vick D J, Goodman Z D, Ishak K G. Squamous cell carcinoma arising in a ciliated hepatic foregut cyst. Arch Pathol Lab Med 1999; 123: 1115-7.
16. Straus T, Osipov V. Ciliated hepatic foregut cyst in a patient with renal cell carcinoma. BMC Cancer 2006; 6: 244.
17. Grizzi F, Chiriva-Internati M, Franceschini B, et al. Immunolocalization of sperm protein 17 in human testis and ejaculated spermatozoa. J Histochem Cytochem 2003; 51: 1245-8.
18. Hornstein A, Batts K P, Linz L J, et al. Fine needle aspiration diagnosis of ciliated hepatic foregut cysts: a report of three cases. Acta Cytol 1996; 40: 576-80.
19. Chatelain D, Chailley-Heu B, Terris B, et al. The ciliated hepatic foregut cyst, an unusual bronchiolar foregut malformation: a histological, histochemical, and immunohistochemical study of 7 cases. Hum Pathol 2000; 31: 241-6.
20. Koletsa T, Tzioufa V, Michalopoulos A, et al. Ciliated hepatic foregut cyst communicating with the gallbladder. Virchows Arch 2005; 446: 200-1.
21. Momin TA, Milner R, Sarmiento JM. Ciliated hepatic foregut cyst of the left hepatic vein. J Gastrointest Surg 2004; 8: 601-3.
22. Kimura A, Makuuchi M, Takayasu K, et al. Ciliated hepatic foregut cyst with solid tumor appearance on CT. J Comput Assist Tomogr 1990; 14: 1016-8.
23. Lim S H, Wang Z, Chiriva-Internati M, et al. Sperm protein 17 is a novel cancer-testis antigen in multiple myeloma. Blood 2001; 97: 1508-10.
24. Chiriva-Internati M, Wang Z, Salati E, et al. Tumor vaccine for ovarian carcinoma targeting sperm protein 17. Cancer 2002; 94: 2447-53.
25. Straughn J M Jr, Shaw D R, Guerrero A, et al. Expression of sperm protein 17 (Sp17) in ovarian cancer. Int J Cancer 2004; 108: 805-11.
26. Grizzi F, Chiriva-Internati M, Franceschini B, et al. Sperm protein 17 is expressed in human somatic ciliated epithelia. J Histochem Cytochem 2004; 52: 549-54.
27. Takeoka Y, Kenny T P, Yago H, et al. Developmental considerations of sperm protein 17 gene expression in rheumatoid arthritis synoviocytes. Dev Immunol 2002; 9: 97-102.
28. Franceschini B, Grizzi F, Colombo P, et al. Expression of human sperm protein 17 in melanophages of cutaneous melanocytic lesions. Br J Dermatol 2004; 150: 780-2.
29. Grizzi F, Gaetani P, Franceschini B, et al. Sperm protein 17 is expressed in human nervous system tumours. BMC Cancer 2006; 6: 23.
30. Bumm K, Grizzi F, Franceschini B, et al. Sperm protein 17 expression defines 2 subsets of primary esthesioneuroblastoma. Hum Pathol 2005; 36: 1289-93.
31. Lea I A, Adoyo P, O'Rand M G. Autoimmunogenicity of the human sperm protein Sp17 in vasectomized men and identification of linear B cell epitopes. Fertil Steril 1997; 67: 355-61.
32. De Jong A, Buchli R, Robbins D. Characterization of sperm protein 17 in human somatic and neoplastic tissue. Cancer Lett 2002; 186: 201-9.
33. Wen Y, Richardson R T, O'rand M G. Processing of the sperm protein Sp17 during the acrosome reaction and characterization as a calmodulin binding protein. Dev Biol 1999; 206: 113-22.
34. Frayne J, Hall L. A re-evaluation of sperm protein 17 (Sp17) indicates a regulatory role in an A-kinase anchoring protein complex, rather than a unique role in sperm-zona pellucida binding. Reproduction 2002; 124: 767-74.
35. Newell A E, Fiedler S E, Ruan J M, et al. Protein kinase A RII-like (R2D2) proteins exhibit differential localization and AKAP interaction. Cell Motil Cytoskeleton 2008; 65: 539-52.
36. Can D W, Fujita A, Stentz C L, et al. Identification of sperm-specific proteins that interact with A-kinase anchoring proteins in a manner similar to the type II regulatory subunit of PKA. J Biol Chem 2001; 276: 17332-8.
37. Can D W, Newell A E. The role of A-kinase anchoring proteins (AKaps) in regulating sperm function. Soc Reprod Feral Suppl 2007; 63: 135-41.
38. Scanlan M J, Simpson A J, Old L J. The cancer/testis genes: review, standardization, and commentary. Cancer Immun 2004; 4: 1.
39. Scanlan M J, Gure A O, Jungbluth A A, et al. Cancer/testis antigens: an expanding family of targets for cancer immunotherapy. Immunol Rev 2002; 188: 22-32.
40. Simpson A J, Caballero O L, Jungbluth A, et al. Cancer/testis antigens, gametogenesis and cancer. Nat Rev Cancer 2005; 5: 615-25.
41. Old L J. Cancer/testis (CT) antigens—a new link between gametogenesis and cancer. Cancer Immun 2001; 1:1.
42. Dadabayev A R, Wang Z, Zhang Y, et al. Cancer immunotherapy targeting Sp17: when should the laboratory findings be translated to the clinics? Am J Hematol 2005; 80: 6-11.
43. Arora S, Matta A, Shukla N K, et al. Identification of differentially expressed genes in oral squamous cell carcinoma. Mol Carcinog 2005; 42: 97-108.
44. Gupta G, Sharma R, Chattopadhyay T K, et al. Clinical significance of sperm protein 17 expression and immunogenicity in esophageal cancer. Int J Cancer 2007; 120: 1739-47.
45. Lubrano J, Rouquette A, Huet E, et al. Ciliated hepatic foregut cyst discovered after kidney transplantation in a hepatitis C virus-infected patient: a report of one case and review of the literature. Eur J Gastroenterol Hepatol 2008; 20: 359-61.
46. Wu M L, Abecassis M M, Rao M S. Ciliated hepatic foregut cyst mimicking neoplasm. Am J Gastroenterol 1998; 93: 2212-4.

What is claimed is:

1. A method for diagnosing ciliated hepatic foregut cysts (CHFC) in a subject comprising the steps of:
(a) obtaining a sample from the subject;
(b) detecting in the sample the presence or absence of a cancer-testis (CT) antigen or its mRNA; and
(d) diagnosing CHFC if the presence of a CT antigen or its mRNA is detected.

2. The method of claim 1, wherein in step (b) an antibody or a fragment thereof specific for a CT antigen is applied to the sample to detect the presence or absence of a CT antigen, and wherein the detection of the antibody-CT antigen complex indicates CHFC.

3. The method of claim 1, wherein the CT antigen is Sperm protein 17 (Sp17), AKAP-associated sperm protein (ASP), or a combination thereof.

4. The method of claim 1, wherein the CT antigen is SP17, ASP, NY-ESO-1, CABYR, TSP50, BORIS, RQCD1, BAGE, SSX, SCP-1, Piwi12, LAGE-1, SSX, AKAP, SCP-1, or combinations thereof.

5. The method of claim 1, wherein the sample is a tissue sample.

6. The method of claim 2, wherein immunohistochemistry or immunofluorescence is employed in step (b).

7. The method of claim 1, wherein after step (d), CHFC in the subject is treated if the CT antigen or its mRNA is detected.

8. The method of claim 7, wherein the treatment comprises removing the cyst(s) by surgery or administering to the subject an agent that attenuates the CT antigen's level or activity.

9. The method of claim 8, wherein the surgery is laparoscopic excision.

10. The method of claim 8, wherein the agent is an antibody or a fragment thereof, an siRNA, an shRNA, an antisense molecule, or a ribozyme.

11. The method of claim 10, wherein the agent is administered systemically.

12. The method of claim 10, wherein the agent is injected into a cyst.

13. The method of claim 1, wherein the subject is additionally diagnosed by ultrasound, computed-tomography (CT), magnetic resonance imaging (MRI), fine needle aspiration cytology (FNAC), immunohistochemistry, surgery, or a combination thereof.

14. A method for diagnosing ciliated hepatic foregut cysts (CHFC) in a subject comprising the steps of:
(a) obtaining a sample from the subject;
(b) detecting in the sample the presence or absence of Sperm protein 17 (Sp17) and AKAP-associated sperm protein (ASP); and
(c) diagnosing CHFC if SP17 and ASP are detected.

15. The method of claim 14, wherein the subject is additionally diagnosed by ultrasound, CT, MRI, fine needle aspiration cytology (FNAC), immunohistochemistry, surgery, or a combination thereof.

16. The method of claim 14, wherein SP17 and ASP are detected by antibodies specific to SP17 and ASP, respectively.

17. The method of claim 14, wherein the sample is a tissue sample.

18. The method of claim 16, wherein immunohistochemistry or immunofluorescence is employed in step (b).

19. A method for diagnosing ciliated hepatic foregut cysts (CHFC) in a subject comprising the steps of:
(a) obtaining a sample from the subject;
(b) measuring the level of a CT antigen, or the level of a CT antigen mRNA, in the sample;
(c) comparing the CT antigen level in the sample, or the level of the CT antigen mRNA in the sample, to the level in a control sample; and
(d) diagnosing CHFC if the CT antigen level in the sample, or the level of the CT antigen mRNA in the sample, is at least 120% of the level in the control sample.

20. The method of claim 19, wherein the CT antigen is detected by an antibody or a fragment thereof specific to the CT antigen.

21. The method of claim 19, wherein the control sample comprises one or more samples from normal healthy subjects.

22. The method of claim 19, wherein in step (d) CHFC is diagnosed, if the CT antigen level or the level of the CT antigen mRNA in the sample is at least 150% of the level in the control sample.

23. The method of claim 19, wherein the CT antigen is Sperm protein 17 (Sp17), AKAP-associated sperm protein (ASP), or a combination thereof.

24. The method of claim 19, wherein the CT antigen is SP17, ASP, NY-ESO-1, CABYR, TSP50, BORIS, RQCD1, BAGE, SSX, SCP-1, Piwi12, LAGE-1, SSX, AKAP, SCP-1, or combinations thereof.

25. The method of claim 19, wherein the sample is a tissue sample.

26. The method of claim 20, wherein immunohistochemistry or immunofluorescence is employed in step (b).

27. The method of claim 19, wherein the subject is additionally diagnosed by ultrasound, CT, MRI, fine needle aspiration cytology (FNAC), immunohistochemistry, surgery, or a combination thereof.
